(19) United States
(12) Patent Application Publication
(10) Pub. No.: US 2024/0041465 A1
(43) Pub. Date: Feb. 8, 2024

(54) SPIRAL-FORMING BALLOON FOR CORONARY SINUS USE

(71) Applicant: INTRATECH MEDICAL LTD., Shefayim (IL)

(72) Inventors: Eran HIRSZOWICZ, Ramat Gan (IL); Keren RESHEF, Herzliya (IL); Alon MAY, Caesarea (IL); Moshe BAREL, Petach Tikva (IL)

(21) Appl. No.: 18/264,535

(22) PCT Filed: Feb. 8, 2022
(86) PCT No.: PCT/IL2022/050159

§ 371 Date: Aug. 7, 2023

Related U.S. Application Data
(60) Provisional application No. 63/146,774, filed on Feb. 8, 2021.

(51) Int. Cl. A61B 17/12

(52) U.S. Cl. CPC A61B 17/12136; A61B 17/12036; A61B 17/1204; A61B 17/12122; A61B 2017/1205; A61M 25/1002

(57) ABSTRACT

A balloon catheter system comprising one or more conduits to which are attached a compliant balloon 84 having a non-helical shape in its deflated state, wherein said balloon is constructed such that is capable of adopting a spiral or helical conformation upon inflation, and wherein the external diameter of the spiral or helical balloon thus formed is in the range of 6-15 mm. The balloon catheter system of the present invention may be used for improving the perfusion of cardiac tissues in a mammalian subject, by means of inflating said balloon within the coronary sinus such that it adopts a spiral conformation, thereby causing partial occlusion of said coronary sinus and causing an elevation of coronary sinus pressure.

### FIELD OF THE INVENTION

The present invention is directed to a device and method that may be used to improve therapeutic outcome in myocardial infarction patients. More specifically, the present invention relates to a balloon device that may be inserted inside the coronary sinus without causing complete occlusion.

### BACKGROUND OF THE INVENTION

Myocardial infarction (heart attack) occurs as a result of a decrease or cessation in blood flow to parts of the heart, thereby causing hypoxic damage to the myocardium. In addition to the immediate and direct effects, a myocardial infarction may also lead to the development one or more severe health problems such as heart failure, cardiac rhythm problems and even cardiac arrest. Myocardial infarction is a very serious risk to health and life and affects a large number of people all over the world. In the year 2015, for example, about 15.9 million myocardial infarctions occurred around the world.

In addition to the physical signs and symptoms that accompany the heart attack, various electrophysiological and biochemical changes also occur, and these may serve as important markers for confirming diagnosis. In this regard, electrocardiogram (ECG) results are of particular benefit, as they can be used to immediately confirm whether elevation of the ST section of the ECG wave is present.

ST elevated myocardial infarction (STEMI) is a very serious form of heart attack during which one of the major coronary arteries is blocked. STEMIs constitute about 25-40% of all heart attacks. Due to the severity of the arterial blockage and the damage caused to the cardiac tissues it is essential to begin treatment of the STEMI as soon as possible after diagnosis. In addition to being given immediate drug treatments and supplemental oxygen, STEMI patients are treated with percutaneous coronary intervention (PCI) techniques such as angioplasty and stenting.

Despite significant advances in the effectiveness of stenting and other PCI modalities over the last few decades, current treatment of STEMI appears to have reached a plateau with regard to its effectiveness. Thus, even after having received mechanical reperfusion by a PCI approach, many patients (˜33%) have impaired or suboptimal myocardial perfusion, resulting in larger infarct size, reduced left ventricular function, congestive heart failure, arrhythmias, myocardial remodeling and mortality.

A need therefore exists for a supplementary treatment method to be used in conjunction with PCI techniques in the treatment of patients presenting with STEMI and other types of acute myocardial infarction. Specifically, such a supplementary method is needed in order to reduce infarct size, reperfusion injury and arrhythmias and to prevent the onset of long-term congestive heart failure.

The present invention meets this need by providing a spiral-forming device and method, as will be disclosed hereinbelow.

### SUMMARY OF THE INVENTION

The present inventors have found that it is possible to construct a spiral-forming balloon having dimensions similar to that of the internal diameter and length of the coronary sinus, and that said balloon, when inflated within the coronary sinus may improve the perfusion of the cardiac tissues in myocardial infarction patients.

Thus, the primary aim of the present invention is to provide a spiral-forming balloon which is capable of being inserted within the coronary sinus, such that upon inflation the external surface of the spiral windings thus formed makes contact with inner wall of said coronary sinus. The spiral conformation of the inflated balloon enables said balloon to act as a semi-occlusive device. That is, its presence within the coronary sinus causes an increase in venous pressure within the sinus, while permitting at least partial blood flow therethrough. This, in turn, leads to an increase in collateral inflow from adjacent non-infarct related coronary arteries that supply the region of the myocardium on the edges of the damaged tissue.

Furthermore, the temporary or partial occlusion of the coronary sinus immediately after stenting was found to restrict the outflow of blood from the coronary veins.

The net effect of the above-described hemodynamic changes caused by the presence of the semi-occlusive balloon is that oxygenated blood is forced deeper into the myocardial tissues, including the oxygen-starved ischemic areas.

The clinical significance of the above-described changes in blood flow in the myocardium caused by inflation of a spiral-forming balloon within the coronary sinus is a significantly improved outcome in patients treated with PCI procedures, such as angioplasty and stenting, following acute myocardial Infarction.

The present invention is thus primarily directed to a balloon catheter system comprising one or more conduits to which is/are attached a compliant balloon having a non-helical shape in its deflated state, wherein said balloon is constructed such that is capable of adopting a spiral or helical conformation upon inflation, and wherein the external diameter of the spiral or helical balloon thus formed is in the range of 6-15 mm. The inner lumen of said balloon is continuous with an inflation lumen of one of the catheter tubes to which it is attached.

In one particularly preferred embodiment of the balloon catheter system of the present invention, the balloon is formed from silicone tubing having an elastic modulus (K) of less than 0.01 N/mm and a percentage elongation greater than 300%.

In another particularly preferred embodiment, the silicone tubing has an elastic modulus (K) of less than 0.007 N/mm and a percentage elongation greater than 300%.

The device of the invention is an over-the-wire balloon catheter comprising a spiral-forming balloon attached at its ends to one or two catheter shafts. A suitable arrangement of the balloon and catheter shaft(s) may be found in the co-owned international patent application which published as WO2008117256, the disclosure of which is incorporated herein in its entirety. Briefly, in its most general form, the spiral-forming balloon catheter of the present invention is a balloon catheter device comprising a tubular compliant balloon that is attached at its distal and proximal extremities to a catheter tube. Upon inflation, the balloon, which is incapable of any significant elongation in a proximal-distal direction (due to its terminal attachment to the catheter shaft), adopts a spiral or helical conformation. It is to be emphasized that in its deflated state, the balloon appears as a conventional, low profile, linear (i.e., non-spiral) sheath surrounding the conduit to which it is attached. It is only during inflation that this linear sheath adopts a spiral conformation. It is to be noted that the ability of the balloon to adopt a spiral or helical conformation is an inherent property of the material used to construct the balloon as well as its absolute and relative dimensions (e.g., diameter, pitch, length etc.). Thus, the balloon of the present invention does not require the use of any ancillary structures such as wires, bands or formers in order to adopt said helical shape upon inflation.

For the purposes of the present disclosure, the terms “proximal” and “distal” are defined from the physician's (or other operator's) perspective. Thus, the term “proximal” is used to refer to the side or end of a device or portion thereof that is closest to the external body wall and/or the operator, while the term “distal” refers to the side or end of a structure that is in an opposite direction to the external body wall and/or operator.

In one preferred embodiment the distal and proximal necks of the balloon are attached to a single catheter conduit. In another preferred embodiment, the distal neck of the balloon is attached to one catheter conduit while the proximal neck thereof is attached to a second conduit, wherein said first and second conduits are arranged such that at least a portion of the shaft of one of the conduits is disposed within the lumen of the other conduit.

In another aspect, the present invention is directed to a method for preparing silicone tubing having an elastic (K) modulus of less than 0.01 N/mm and a percentage elongation greater than 300%, comprising the steps of providing standard medical-grade silicone tubing, repeatedly stretching the silicone tube to a length in the range of about 500-600% of its original length, and inflating said stretched tube such that it attains an external diameter that is at least twice its original diameter.

Additionally, the present invention encompasses a method for improving the perfusion of cardiac tissues in a mammalian subject, wherein said method comprises the steps of providing a balloon catheter according to any one of the preceding claims, introducing said balloon catheter into the subject's venous system over a guidewire, advancing said catheter until the balloon becomes located within the coronary sinus, inflating said balloon such that it adopts a spiral conformation, thereby causing partial occlusion of said coronary sinus for the desired length of time, and then completely deflating said balloon and withdrawing the catheter from the subject's vasculature.

In one preferred embodiment of this aspect of the invention, the “desired length of time” for retaining the balloon in its expanded state within the coronary sinus is in the range of 60-90 minutes. However, in other embodiments, this duration can be either shorter or longer than the stated range, as required and determined by the clinician, without deviating from the scope of the method of the present invention.

In one preferred embodiment of this aspect of the invention, the method is used in subjects following myocardial infarction to improve the therapeutic outcome of percutaneous coronary intervention techniques.

In one preferred embodiment, the subject of the above-disclosed method is a human subject. In other embodiments, the method is used for veterinary procedures in non-human mammalian subjects.

### BRIEF DESCRIPTION OF THE DRAWINGS

FIG. 1 illustrates a typical spiral-forming balloon of the present invention in its deflated state, attached to a catheter shaft.

FIG. 2 is a photograph depicting a balloon catheter of the present invention attached to a catheter tube, following expansion into its spiral shape, within the confines of a plastic tube.

FIG. 3 shows a length of silicone tubing placed within a balloon tube stretch device.

FIG. 4 depicts the same length of tubing shown in FIG. 3 after it had been removed from the stretch device and placed within a balloon inflation device.

FIG. 5 graphically depicts the applied force-strain relationship curves for 10 samples of untreated silicone tubing.

FIG. 6 graphically depicts the applied force-strain relationship curves for 10 samples of silicone tubing following the pre-treatment of said tubing, according to the procedure of the present invention.

FIG. 7 illustrates the non-spiral shape adopted by a balloon constructed conventional, untreated length of silicone tubing following its inflation, when said balloon is attached at both of its ends to a catheter shaft.

FIG. 8 illustrates the component parts of one embodiment of the catheter system of the present invention.

FIG. 9 illustrates a typical introducer sheath and loader that may be used in conjunction with the catheter system of the present invention.

### DETAILED DESCRIPTION OF PREFERRED EMBODIMENTS

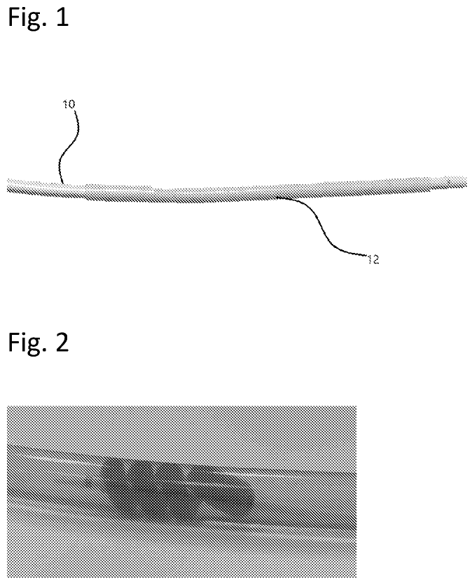

Certain preferred embodiments of the present invention will now be described in relation to the appended figures. Thus, in its deflated state, as shown in FIG. 1, the balloon 12 of the present invention is in the form of a tube of compliant material having either a uniform wall thickness or with a wall thickness which varies along its length. As shown in this figure, the collapsed balloon 12 attached at each of its ends to the outer surface of a rigid or semi-rigid catheter shaft 10. The attachment of the balloon to the catheter conduit may be achieved using any of the standard bonding techniques and materials well known in the art, for example adhesion using biocompatible glues such as silicone glue.

FIG. 2 photographically depicts a typical example of a balloon of the present invention attached to a catheter tube, as described above, after it has been expanded within a plastic tube (thereby simulating expansion within a vein). As shown, the balloon adopts a spiral or helical conformation around said catheter tube upon inflation. The spiral formation occurs due to the fact that since the balloon is bound at both its ends, its longitudinal elongation is restrained. Provided that the balloon is made of a compliant material possessing certain physical parameters (as will be discussed hereinbelow) it will undergo uniform buckling thereby adopting a spiral or helical form during inflation.

In order for the balloon to be suitable for use within the coronary sinus, it is necessary for its outer diameter, in its inflated state, to be the same or larger than that of the coronary sinus itself. Since the coronary sinus, in healthy individuals, has an internal diameter of about 1 cm [D'Cruz, Shala & Johns (2000) “Echocardiography of the coronary sinus in adults”; Clin. Cardiol. 23: 149-154], the balloon of the present invention would generally need to expand to an external diameter in the range of in the range of approximately 6-15 mm. This is much larger than the diameter of prior art spiral-forming balloons (e.g., as described in co-owned WO2008117256), which when used in the cerebral vasculature require an external diameter of about 2-4 mm. It may therefore be appreciated that the spiral-forming balloon of the present invention, i.e., one which is suitable for use within the coronary sinus is far larger in diameter than any of the prior art balloons, which were designed for use within much smaller blood vessels.

Using different wall thicknesses or different materials the shape of the spiral/helix and the inflation sequence can be controlled.

Typically, the compliant balloon will have a length in the range of 20 mm to 60 mm and a wall thickness in the range of 0.15 mm to 0.5 mm. It should be emphasized that the preceding dimensions (and all other dimensions that appear herein) are exemplary values only and should not be construed as limiting the size of the presently disclosed device in any way.

The general embodiment of the balloon catheter of the present invention that is described hereinabove comprises a single catheter conduit to which the compliant balloon is attached. However, it is to be recognized that many other catheter conduit conformations may also be used in the present invention. For example, instead of the single-conduit system, the device of the present invention may have a two-conduit conformation, with (for example) the proximal neck of the balloon being attached to the outer surface of an outer conduit, while the distal neck thereof is attached to the outer surface of an inner conduit that is disposed within the lumen of said outer conduit. In this type of conformation, the inner conduit will generally extend beyond the distal end of the outer conduit. The device of the present invention may also comprise one or more conduits having multiple lumens (e.g., bi-lumen catheters) where the additional lumens may be used for a variety of purposes, including the passage of guidewires or instrumentation of various types.

The compliant balloon may be inflated by introducing a pressurized inflation media via an inflation fluid port that is in fluid connection with a source of pressurized media and a pumping device or syringe. In the case of a single conduit catheter, the inflation media passes through openings in the wall of the catheter shaft located between the proximal and distal attachment points of the balloon. In the case of a dual (inner-outer) conduit conformation, as described above, the inflation media passes via an inflation fluid lumen formed between the inner wall of the outer conduit and the outer surface of the inner conduit. The pressure in the balloon when fully inflated with an expansion medium such as saline or a contrast medium is in the range of 0.5-4 atmospheres, and often in the range of 1.5-2 atmospheres.

One technical problem encountered by the present inventors is that it was not possible to construct spiral-forming balloons of the size required for coronary sinus implantation, using standard medical-grade silicone (or other) polymers. Indeed, it was found that the use of these standard materials with balloons of the required size resulted in an inability to inflate into a regular spiral or helical form. However, it was found by the present inventors that only polymers having novel and highly specific physical characteristics could be used to manufacture balloons having an inflated external diameter in the range of 6-15 mm which are capable of adopting a spiral conformation upon inflation. Thus, it was found that in order for a silicone tube to form a spiral balloon having an inflated external diameter of the desired size it was necessary to prepare a tube having most or all of the same physical properties of standard, off-the-shelf medical-grade silicone rubbers, except for a significantly reduced elastic (‘K’) modulus.

Without wishing to be bound by theory, it is believed that since the reduced elastic modulus of the balloons used in the present invention enables less inflation force to be applied during inflation to produce the same degree of volumetric expansion, the inflation process becomes much more homogenous, thereby permitting all regions of the internal space of the tube to be expanded at the same time. In this way, the formation of local ‘blisters’ or other non-uniform areas of expansion can be prevented, thereby allowing the balloon, attached at both of its ends to the catheter tube, to expand into a regular spiral conformation.

Thus, in one preferred embodiment, the compliant balloon of the system of the present invention is formed from silicone tubing having a significantly reduced elastic (K) modulus, while some or all of the other physical parameters of said tubing (e.g., its ability to undergo elastic elongation, tensile strength and so on), have similar values as for standard medical-grade silicone tubing.

In one preferred embodiment, the K modulus of the silicone tubing used to prepare the balloon has a value less than 0.01 N/mm.

In one preferred embodiment, the K modulus of the silicone tubing used to prepare the balloon has a value less than 0.007 N/mm.

In one preferred embodiment, the K modulus of the silicone tubing used to prepare the balloon has a value of approximately 0.005 N/mm.

Preferably, the percentage elongation of said silicone tubing (i.e., the ability of said tubing to undergo elastic elongation) is greater than 100%. More preferably, said percentage elongation is greater than 300%. More preferably still, this parameter has a value in the range of 300% to 800%. In one preferred embodiment, the percentage elongation of the silicone tubing is approximately 600%.

In one embodiment, the tear resistance of said silicone (as determined by the ASTM D-624 protocol) is at least 17 N/mm.

In one embodiment, the tensile strength of said silicone is at least 8 MPa.

Thus, in order to manufacture the spiral-forming balloon of the present invention, having the ability to adopt a spiral conformation upon inflation when bound at both ends to a catheter tube, said balloon must be produced from a silicone tube having the physical properties defined hereinabove. To the best of the inventors' knowledge, no such material is currently commercially available. However, it has now been found that by means of applying a certain type of pre-treatment to a standard medical-grade silicone material having a K modulus of at least 0.01 N/mm (with a typical value of around 0.04 N/mm) it is possible to obtain a silicone tube having the desired physical parameters defined hereinabove. Briefly, the pre-treatment method of the present invention comprises repeatedly stretching the silicone tube to a length in the range of about 500-600% of its original length and then inflating said stretched tube such that it attains an external diameter that is at least twice its original diameter, and preferably about 2-3 times its original diameter.

In one preferred embodiment the silicone tube that is pre-treated as described hereinabove has an external diameter (i.e., before pre-treatment) in the range of 1-3 mm.

The details of one non-limiting example of this pre-treatment method are provided in Example 1, hereinbelow.

In practice, the spiral-forming balloon catheter of the present invention is introduced into the venous system over a guidewire (with the aid of an introducer sheath and/or guiding catheter) via a convenient access point, for example the femoral, jugular or brachial veins. Once the balloon has been steered into the coronary sinus, it is then inflated using an appropriate inflation medium. Upon full inflation, the balloon adopts a spiral or helical conformation and is self-stabilizing within the coronary sinus by virtue of the forces applied by the inflated spiral balloon on the inner wall of the vessel.

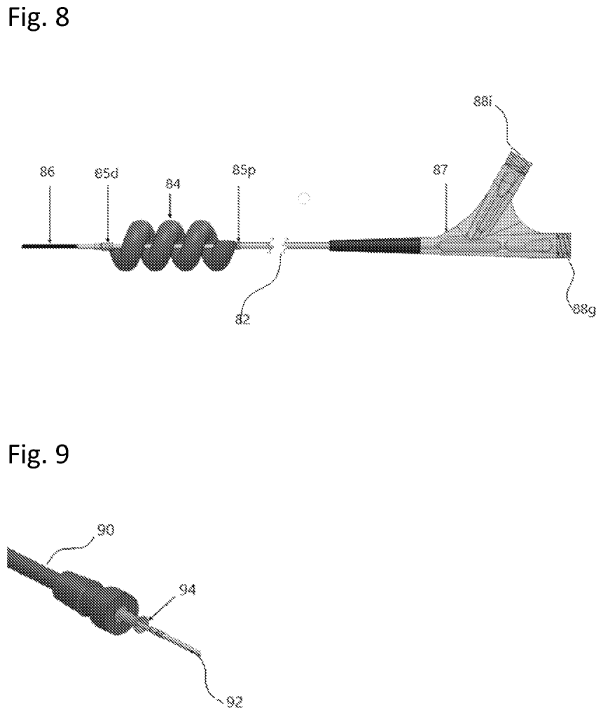

FIG. 8 illustrates one embodiment of the catheter system 80 of the present invention that is suitable for use in introducing the spiral-forming balloon into the coronary sinus and removing same therefrom at the end of the procedure. The figure shows the proximal and distal sections of the catheter tube 82. A spiral-forming balloon 84 is shown (in its expanded state) wound around the external wall of said catheter stube. Radio-opaque distal and proximal markers 85d and 85p are present on the external surface of the catheter tube next to, respectively, the distal and proximal attachment points of said balloon to said catheter tube. Catheter tube 82 ends distally in a stylet 86. The proximal extremity of the catheter tube is connected to, and in fluid connect with, a hub unit 87, which comprises two separate hubs: an inflation hub 88i and a guidewire hub 88g. The system shown in FIG. 8 is only one of several different systems that may be used to work the present invention, and is characterized by the following dimensions:
catheter length 100 cm
0.035″ OTW
Compatible with a 9F (or larger) introducer sheath
Balloon length 35 mm
Expanded balloon diameter range: 8-10 mm
Crossing profile: 2.42 mm

It is to be emphasized that these parameters relate to one specific embodiment, and that the scope of the present invention is not limited to said parameters.

FIG. 9 illustrates an introducer sheath 90, which may be used to together with a suitable loader, such as funnel-shaped loader 94, in order to facilitate passage of the guidewire and balloon catheter 92 through the subject's vasculature (e.g., via puncture of the jugular or femoral veins) into the coronary sinus.

In general terms, the catheter system of the present invention is introduced into the coronary sinus of a patient, and removed therefrom, by means of the following steps:
1. Cannulation of the jugular or femoral veins using an introducer with a diameter of 9 Fr or larger.
2. Location and marking of the distal balloon catheter positioning point using fluoroscopic imaging.
3. Introduction of a stiff guidewire (e.g., 0.035″ diameter) into the distal portion of the coronary sinus.
4. Using a loader inserted into the proximal portion of the introducer sheath (i.e., the introducer sheath valve), advance the balloon catheter of the present invention into the coronary sinus, under fluoroscopic guidance, positioning the distal marker band a few millimeters distal to the distal positioning point (determined in step 2, above).
5. Remove the loader from the introducer sheath valve.
6. Remove the guidewire and connect a pressure monitor to the guidewire hub.
7. Record the pre-inflation coronary sinus pressure (using the pressure monitor fitted in step 6).
8. Position the distal marker band at the distal positioning point.
9. Inflate the balloon with a pre-calibrated inflation fluid volume, introduced via a syringe connected to the inflation hub, in order to achieve the desired inflated diameter (the same or slightly larger than the coronary sinus diameter).
10. Record the post-inflation coronary sinus pressure, using the monitor connected to the guidewire hub.
11. Maintain the balloon in its inflated state for the desired treatment duration (e.g., 60-90 minutes).

### Device Removal:

12. Re-introduce the guidewire into its lumen via a loader placed in the introducer sheath valve.
13. Completely deflate the balloon under fluoroscopy, by applying negative pressure to the syringe placed in the inflation hub.
14. Withdraw the catheter following complete balloon deflation.

The above procedure steps are given for purposes of exemplifying the invention; many different alterations in the procedure may be performed by the skilled clinician, in accordance with her/his experience, without deviating from the scope of the present invention.

The spiral-forming balloon of the present invention possesses the following advantageous properties:
A) Upon inflation within the coronary sinus it causes only partial occlusion of that vessel, thereby permitting continued venous drainage from the heart. This occurs by virtue of the spiral space formed between the continuous spiral groove on the external surface of the balloon and the wall of the coronary sinus, thereby permitting continued transfer of fluid along the sinus.
B) The balloon is self-anchoring within the coronary sinus.
C) The balloon may be manufactured in more than one size in order to accommodate differences in anatomic diameter and length of the coronary sinus.
D) The balloon may be manufactured with different numbers of pitch windings per unit length, in order to achieve a desired pressure increase and/or degree of partial occlusion within the coronary sinus.

The present invention will be described in more detail in the following Examples which is illustrative only and does not limit the scope of the invention in any way.

### EXAMPLES

### Example 1

### Illustrative Example of Silicone Tube Pre-Treatment that May be Used to Produce a Spiral-Forming Balloon for Use in the Present Invention

For the purpose of this Example, a medical-grade silicone tube (manufactured from liquid silicone rubber) having an internal diameter of 1.25 mm and an external diameter of 1.75 mm was used.

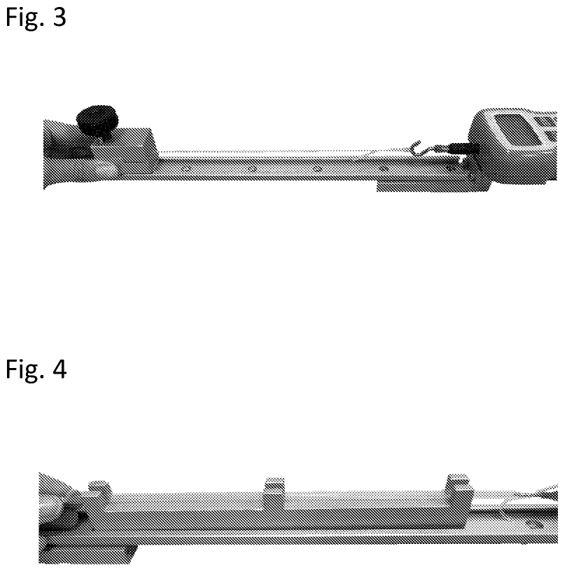

A 12 cm length of the silicone tubing was attached to a sliding balloon tube stretch device, shown in FIG. 3. The open end of the tube was inserted into the fixture clamp on one end of the device (on the left side of FIG. 3), while the other end of the tube was looped and threaded over the force gauge hook (on the right side of FIG. 3). The fixture was then slid (towards the left in the figure) such that the tube was stretched. When the tube color became white the force on the tensile force gauge meter of the stretching device was read and noted. This stretching procedure was then repeated until the tensile force at the endpoint was such that the tube turned white.

Next, the tube was removed from the stretch device and its open end was secured over a needle connected to a disposable syringe. The tube was then secured in the inflation device depicted in FIG. 4. The tube was then inflated gently with air using the syringe until the tube color became white and its outer diameter reached a value of about 2-3 times its original value. The tube was then deflated, and this procedure repeated three times.

### Example 2

### Comparative Study of the Elastic Modulus (K) of the Modified Silicone Tube of the Present Invention Vs. a Standard Untreated Tube

Method:

10 cm lengths of the same medical-grade silicone tubing used as the starting material in the preparative method described in Example 1 were prepared. The same number of identical length tubes following the pre-treatment procedure of Example 1 were also prepared.

Each tube was then tested by connecting it to a tensile strength testing machine provided by the Testometric Company Ltd. (Rochdale, UK), and a pull test conducted with the assistance of the WinTest™ software supplied by the same company. Ten pieces of pre-treated silicone tubing, and ten pieces of untreated silicone tubing were tested using this device. The length of each tubing sample tested was 60 mm, and the pull test was carried out at a linear test speed of 100 mm/minute, in the absence of any applied pretension.

Results:

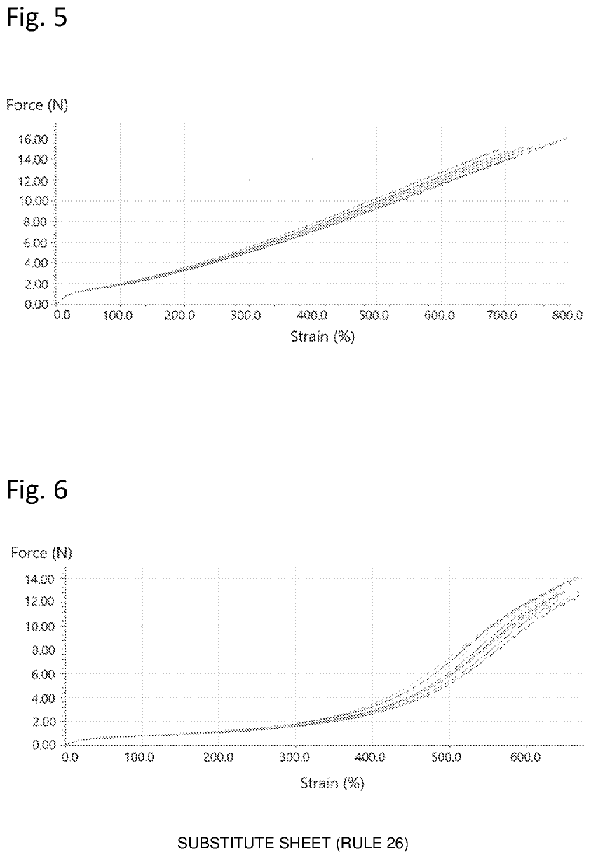

FIG. 5 graphically presents the results for the applied force-strain relationship for the 10 samples of untreated silicone tubing. Each line on the graph represents one of the 10 samples. The range of the graph relevant for the clinical usage of such tubing as intravascular balloons would be from a strain (x-axis) value of 0 to about 400. Similarly, FIG. 6 provides the comparable results for silicone tubing following the pre-treatment procedure described in Example 1, hereinabove. It may be readily seen that the slope of the stress-strain curve is significantly lower in the pre-treated samples (FIG. 6) than in the untreated samples (FIG. 5). The mean results for the K modulus obtained from the test device software for the pre-treated and untreated silicone tubing samples were 0.005 N/mm and 0.04 N/mm for the untreated samples.

These results indicate that the pre-treated silicone tube samples have a significantly reduced elastic (i.e., K) modulus.

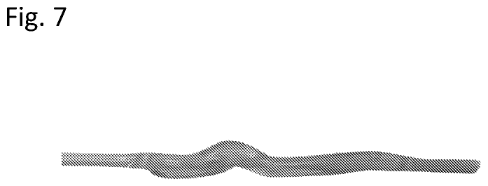

Finally, the present inventors have found that the altered physical properties of the pre-treated silicone tubing (i.e., significant reduction in the K modulus while maintaining most or all of the other physical properties unaltered) results in the ability of said tubing to form a spiral or helical balloon when attached by both of its ends to a catheter tube and then inflated. One example of such a spiral-forming balloon constructed from pre-treated silicone, following inflation, is shown in FIG. 2. Conversely, when conventional, off-the-shelf untreated medical silicone tubing is used, and inflated under identical conditions, it proves impossible for said tubing to expand into a regular spiral or helical shape. Rather, as shown in FIG. 7, the resulting inflated tubing forms a few irregular blister-like areas.

### Example 3

### In Vivo Animal Study—Insertion of Balloon Catheter Device of the Present Invention into The Porcine Coronary Sinus

Aim:

The aim of this study was to evaluate the performance and safety of the balloon catheter system of the present invention in pigs having normal cardiovascular physiology.

Methods:

A group of five healthy female domestic pigs weighing approximately 50-55 Kg were selected for the study.

The animals were treated with antiplatelet therapy (Plavix, Aspirin) for one day prior to the procedure and on the morning of the procedure. Heparin administration was maintained throughout the procedure.

The balloon catheter was inserted via jugular vein access using an introducer sheath, and the balloon was inflated to a 1:1 vessel/balloon diameter ratio. The balloon was maintained in its inflated (i.e., spiral) conformation for 90 minutes, and coronary sinus pressure was measured both before inflation and immediately after inflation.

After the 90-minute period, the balloon was deflated, the catheter removed from the animal and the jugular vein access site closed with sutures.

During balloon inflation, the following measurements and observations were recorded: coronary sinus pressure distally to the balloon, blood drainage through the inflated balloon was confirmed in two animals, balloon diameter during inflation time within the coronary sinus was measured immediately following inflation and soon after balloon removal. The animals' vital signs and systemic arterial pressure were continuously monitored.

At the study termination, echocardiographic assessments were performed in order to look for the changes in left ventricular wall thickness, contractility and hemodynamic parameters.

Results:

All procedures were successfully conducted with satisfactory positioning of the balloon within the coronary sinus.

The echocardiography assessments (at three timepoints: prior to balloon insertion, after balloon removal and at 30 days) demonstrated no change in LV wall thickness, contractility or hemodynamic parameters.

In four of the five animals in the group, the balloon diameter was maintained throughout inflation. Continuous coronary sinus venous drainage was seen during balloon inflation.

Elevation of coronary sinus pressure was seen immediately following balloon inflation, and remained stable throughout the procedure, as shown in the following table:

| | | Coronary sinus | | |
|---|---|---|---|---|
| | | (CS) pressure | | |
| | | immediately | CS pressure | CS pressure |
| | Baseline | following | 60 minutes | 90 min |
| Animal | pressure | inflation | post- | post |
| no. | (mmHg) | (mmHg) | inflation | inflation |
| 1 | 7 | 9 | 9 | 7 |
| 2 | 7 | 10 | 8 | 8 |
| 3 | 5 | 7 | 6 | 5 |
| 4 | 9 | 13 | 13 | 10 |
| 5 | 11 | 16 | 14 | 15 |

It is concluded from this animal study that the balloon catheter device of the present invention may be inserted and inflated within the coronary sinus without any undue technical difficulty. Furthermore, the absence of any deterioration in any of the echocardiographic parameters indicates that insertion and use of the device in the coronary sinus does not cause any significant cardiac trauma. Finally, inflation of the balloon resulted in the desired elevation in coronary sinus pressure, indicating its suitability for its intended therapeutic use.

1. A balloon catheter system comprising one or more conduits to which is/are attached a compliant balloon having a non-helical shape in its deflated state, wherein said balloon is constructed such that is capable of adopting a spiral or helical conformation upon inflation, and wherein the external diameter of the spiral or helical balloon thus formed is in the range of 6-15 mm.

2. The balloon catheter system according to claim 1, wherein the balloon is formed from silicone tubing having an elastic modulus (K) of less than 0.01 N/mm and a percentage elongation greater than 300%.

3. The balloon catheter system according to claim 2, wherein the silicone tubing has an elastic modulus (K) of less than 0.007 N/mm and a percentage elongation greater than 300%.

4. A method for preparing silicone tubing having an elastic (K) modulus of less than 0.01 N/mm and a percentage elongation greater than 300%, comprising the steps of providing standard medical-grade silicone tubing, repeatedly stretching the silicone tube to a length in the range of about 500-600% of its original length, and inflating said stretched tube such that it attains an external diameter that is at least twice its original diameter.

5. A method for improving the perfusion of cardiac tissues in a mammalian subject, wherein said method comprises the steps of providing a balloon catheter according to claim 1, introducing said balloon catheter into the subject's venous system over a guidewire, advancing said catheter until the balloon becomes located within the coronary sinus, inflating said balloon such that it adopts a spiral conformation, thereby causing partial occlusion of said coronary sinus for the desired length of time, and then completely deflating said balloon and withdrawing the catheter from the subject's vasculature.

6. The method according to claim 5, wherein said method is used in subjects following myocardial infarction to improve the therapeutic outcome of percutaneous coronary intervention techniques.

7. The method according to claim 5, wherein the subject is a human subject.
